(19) United States
(12) Patent Application Publication
(10) Pub. No.: US 2005/0050831 A1
(43) Pub. Date: Mar. 10, 2005

(54) Compound post union frame

(76) Inventor: Charles Dean Lyon, Mount Calm, TX (US)

Correspondence Address: Charles Dean Lyon, 1324 LCR 120, Mount Calm, TX, 76673-3016, US

(21) Appl. No.: 10/826,181

(22) Filed: Apr. 16, 2004

Related U.S. Application Data
(60) Provisional application No. 60/500,301, filed on Sep. 5, 2003.

(51) Int. Cl. C12P 21/06; E04H 12/00

(52) U.S. Cl. 52/653.1

(57) ABSTRACT

A Compound Post Frame (28-80) with securing devices (46A). It provides a means to united small post, pipe, rods or tubing (46C) in marriage to form a strong single Compound Post (80). Other items can be attach easily and the post replaces any post of any material.

### CROSS-REFERENCE TO RELATED APPLICATIONS

This application is entitled to the benefits of the Disclosure Ser.#536671 filed Aug. 18, 2003. This application is entitled to the benefits of Provisional Patent Application Ser.#60/500301 filed Sept. 5, 2003. This invention uses the transmission of my co-pending application, Disclosure Ser.#541450 filed Nov. 6, 2003.

Compound Post Union Frame for the purpose of having a means to securely fasten elongated objects together to form a single post.

### STATEMENT REGARDING FEDERALLY SPONSORED RESEACH OR DEVELOPMENT

NOT APPLICABLE

### SEQUENCE LISTING

NOT APPLICABLE

### BACKGROUND OF THE INVENTION

The present invention relates to construction of a frame 28 to join two or more post, pipe, rods or similar items 46C into a super strong durable post 80. The Frame 28 makes it easy to attach items to the Frame and exposes individual post 46C for easy attachment of items to them. This Compound Post 80 can be used in the place of any post of any material for enclosure fences, building support post, power poles, light support, observation towers, barricades, and antenna.

Compound Post 80 will have many possible uses and variants to describe even the ones already mention would be extensive. Descriptions, design, use and installation describe here will be for enclosure, exclusion fences. Fence T-post 46C will represent all of the elongated items possible for use in horizontal or vertical positions.

The present invention relates to fence post construction 80, and more particularly to a T-post 46C fencing unit. Another aspect of the present invention is a lateral bracing arrangement or assemblage that may be used for a comer fence assembly utilizing at least two T-post 46C fencing units described herein. The present invention further relates to a method for forming a braced fencing arrangement, a rail fence assembly, a puzzle method, and a post extender method.

Fencing patents have been numerous for over a 100 years proving that a need for improved fencing is important. The T-post 46C and barbwire have became the predominate choice. The T-post 46C alone is lacking in strength and stability for building a long life fence. The compound post 80 presented here has strength in all directions and is easily braced in all directions if so desired. The Compound Post 80 is very serviceable when made of readily available T-post 46C. I have personal experience of T-post 46C still in use after 50 years. Wooden post and even steel pipe rarely last this long, and both may contain hazards material.

U.S. Pat. No. 6,536,745 issued to Roark, Harold Dean on Mar. 25, 2003 relates the fencing problems very well. It has a post and brace design that does brace in the line of the fence. This Compound Post 80 does not retain the weak points of a single T-post the way his patent does. The Compound Post 80 has a untied strength in all directions and has fewer and less expensive parts, it is more securely lock together for long term use and abuse.

U.S. Pat. No. 0,066,995A1 issued to Collins,Charles R. on Apr. 10, 2003 is like all previous designs, braced in the line of fences, weak to ninety degree pressure, complicated to build and assemble. The Compound Post 80 has the strength of multiple T-post 46C and with compound frame of choice can be assembled with readily available materials.

U.S. Pat. No. 6,443,433B1 issued to Auldridge, Douglas on Sep. 3, 2002 has a capable way of making a light duty rail fences that retains the weakness of the single T-post except in the direction the fence runs. The Compound Post 80 has the lateral strength and can be assembled with any of several styles of Compound Frame 28.

U.S. Pat. No. 5,395,093 issued to Chrisman, Lawrence C. on Mar. 7, 1995 has a patent for a T-post height extender but is limited to a single T-post and the weakness inherit. The Presented Compound Frames 28 can be made into a extender by adding more T-post 46C positions with unlimited horizontal length and height limited by stability only.

U.S. Pat. No. 3,119,471 issued to Turner B. R. on Jan. 28, 1964 has a tower design that has to be pre-welded post to braces. The presented Compound Frame 28 can be assembled in the field making shipping of materials more compact. T-post and cement reinforcements bar 46C are being produced in recycle steel foundries wide spread in the US.

U.S. Pat. No. 826,996 issued to Cooke, C. C. on Jul. 24, 1906 has a telegraph pole design, but is more complicated, requires more parts and is more difficult to assemble than the Compound Frame 28 assemble being presented.

U.S. Pat. No. 6,000,682 issued to Lechtenbohmer, Hans Norbert on Dec. 14, 1999 has a patent with mulitiple rods for a compound fence post but appears to be designed for wood plank fence only and is extremly complicated and costly.

U.S. Pat. No. 6,585,234 issued to Berto; Joseph J. Jul. 1, 2003 has a device attaching too a T-post 46C. Berto's patent holds pvc 46C and wire. The Post Frame 28-80 though different will do all that Berto's does. The Compound Post Union Frame 28-80 will also have bracing capabilities and strength in all directions not provided with his. The Compound Post Union Frame 28-80 will also be a height extender 44E if needed.

### BRIEF SUMMARY OF THE INVENTION

The Compound Post 80 makes possible an all metal fence of uniform appearance. Said fence can be made that will have minimal damage by fire. Compound Posts 80 are strong for force pull points and where fence line changes direction. The Compound Post 80 is strong in all directions, creating a more lean proof fence. Gates, barbwire and other items attach easily. Digging post-holes is not required and firming time for stress post is eliminated. The material to build is readily available. Arsenic treated wooden post have been found to be dangerous. Wooden post often rot or burn. Pipe is expensive and used pipe often have contaminates. Rail fences with Compound Post Union Frame 28 can have a rail at each Frame 28 level made of T-post, Pvc Pipe, cement reinforcement rod or other items 46C. The Compound Post 80 with top Compound Post Union Frame 28 having additional post positions 44E can be extended. The round Frame 62-80 can form hunting or observation towers80, and other revolving items, such as lights. Compound Post Union Frame 62-80 can be fitted with one or several 360 degree gates for livestock work. The Compound Post Union Frame 62-80 is a good design for use with antennas. Materials, size, and shape of both Frame 28 and connected items 46C may be varied for any reason.

### BRIEF DESCRIPTION OF THE DRAWINGS

FIG. 1. A through F are connectors 50. 1i is a connector for Track 66A FIG. 7B to Compound Post 80. 1 G-H and J are prior art, as samples of items used.

FIG. 2. A diagonal view of square Frame 28-54 with an engaging means on three sides. Connectors are plate 50B.

FIG. 3. A diagonal view of square Frame 28-56 with engaging means on three sides. Connectors are rods 50A2

FIG. 4. A diagonal view of square Frame 28-58 with engaging means on three sides. Connectors are 34-50E cut from plate 48.

FIG. 5. A diagonal view of a square one plate 48 Frame 28-52 with engaging means on three sides. Connectors are channel 50C.

FIG. 6A. Side view of one channel bar Frame 28-72 with engaging means 44 on channel sides and both ends.

FIG. 6B. Side view of two Compound Post Union Frame 28-72 joined to form a T with engaging means 44 on channel sides and exposed ends.

FIG. 6C. Side view of three channel bar Frame 28-72 joined to form a capital I with engaging means 44 on three channel sides and four exposed ends.

FIG. 6D. Side view of four equal length channel bar Frame 28-72 joined at the ends to form a rectangle with engaging means on four channel sides. Four engaging means 44E add extension capabilities as an example of extender 44E.

FIG. 7A. View of round Frame 28-62 with engaging means for brace, six support post 46C, and optional a three hundred and sixty degree turn attachment.

FIG. 7B. View of round track 66A used for revolving attachments on lower Compound Post 80-62 assemblies.

FIG. 7C. View of upper arm 70 with top engaging means from Compound Post Union Frame 80-62 to attachments.

FIG. 7D. View of yoke 68 with low engaging means from track 66A to attachments.

FIG. 8. Side view of trapezoid Frame 28-60 with engaging means on two sides.

FIG. 9. Corner view of assembled two rail fence corner or barbwire fence braced corner of Compound Post 80.

### DETAILED DESCRIPTION OF THE INVENTION

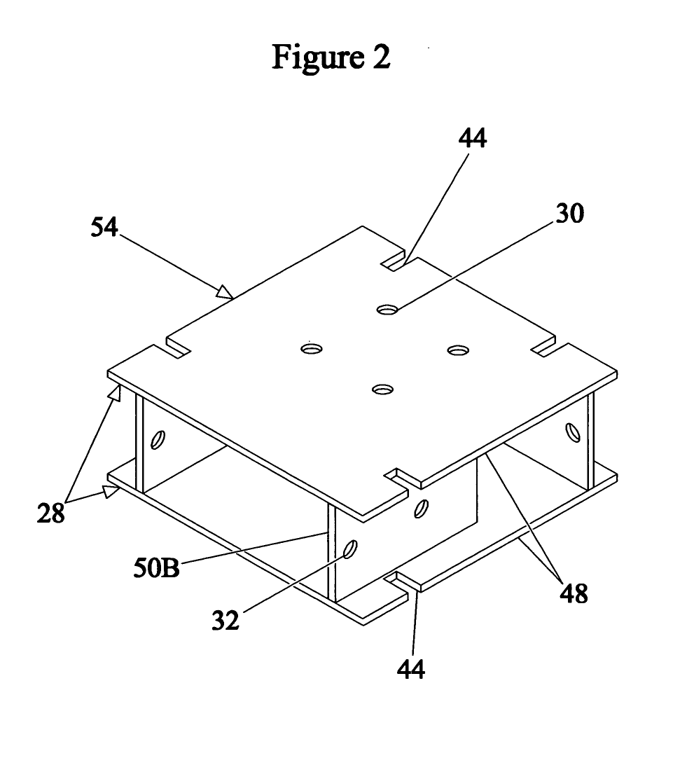

28. Frame FIGS. 2-3-4-5-6-7-8-9

Compound Post Union Frame 28 can be round 62, trapezoid 60 square 52-54-56-58-78, bar 72-74-76-78 or rectangle (not shown in flat material) but is made by extending a square between, the side with two cutouts 44 and the opposite side without cutouts 44. A compound post frame 28 will hold small post 46C together to form a larger stronger post 80.

30. Holes Frame 28 FIGS. 2-3-4-5-7-9

Holes in all frames 28 (except channel bar 72-74-76-78 on these models holes are optional the U-bolt and bracket 46A-B surrounds bar 72) to accept insertion of U-bolts 46A, for attaching braces of T-post, Pipe or Cement reinforcement bar 46C, from one compound post 80, to another compound post 80, or to attach a gate or other items to the compound post 80. The holes 30 can be arranged so that two clamps 46A can hold brace post 46C in all directions.

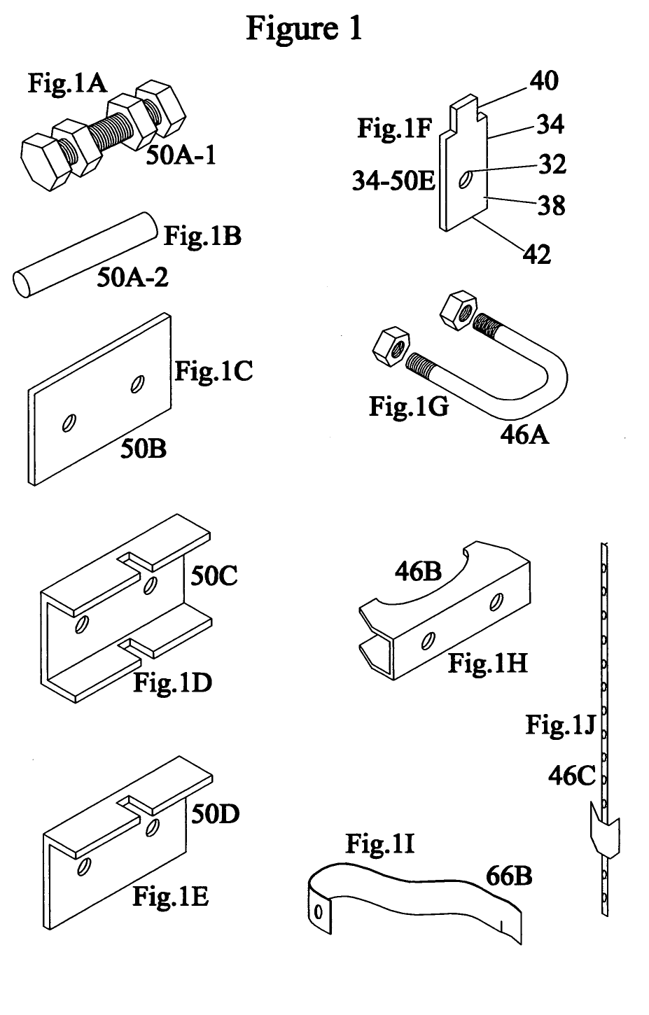

32. Holes in clamp connectors FIGS. 1-2-4-5-6-7-8-9

Holes in clamp connectors FIG. 1F 34-50E, 50-B-C-D for U-bolt clamps 46A. To attach individual post 46C to post frame (28).

34. Bottle shaped connector FIGS. 1-4

Bottle shaped 50E connector 34-50E formed by cut and bend 42 out, 36 leaving one side attached to be bent (at point 42) out to connect two identical cutout 34-50E plates 48 to form a frame 28 for connecting post 46C to form a compound post 80.

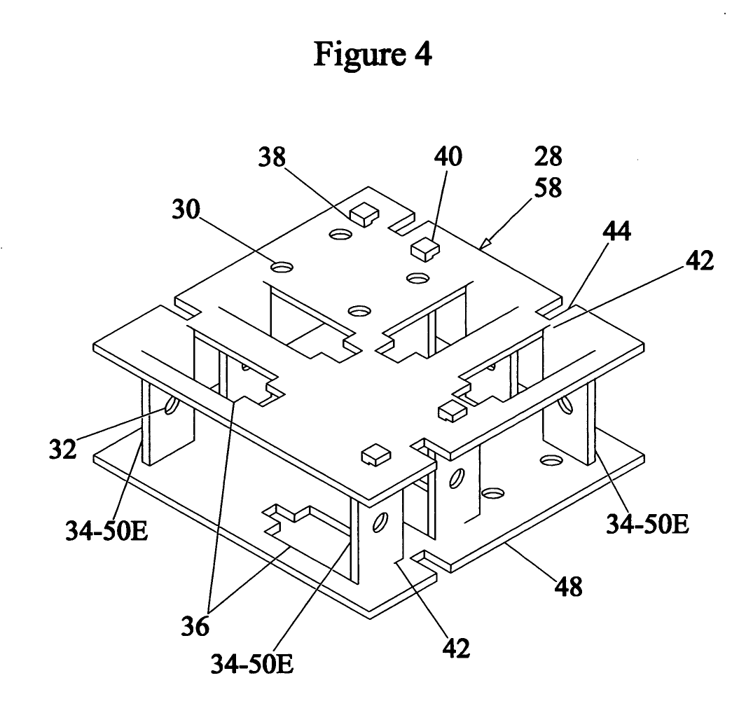

36. Holes FIG. 4

Holes 36 not used where a connector 34-50E has been cut and bent 42 out.

38. Rectangle cutouts FIG. 4

rectangle cutouts to accept the protruded top 40 of bottle shaped cutout 34-50E.

40. Protruded top of bottle shaped connector 34-50E FIG. 1F-4

Protruded top of bottle shaped cut 34 and bend 42 out 34-50E made to insert into cutout 38 to attach and separate plates 28.

42. Bend out point of connector FIG. 1F-4 Bend out point of connector 50E, cutout 34.

44. Rectangle cutouts FIGS. 1D and E-2-3-4-5-6-7-8-9

Rectangle cutouts on the edge of all frames 28 and connectors 5OC-D to accept individual post 46C to form a compound post 80. Present drawings and descriptions or for a cutout 44 to accept the unnoduled side of a enclosure fence type T-post 46C, but represent all items that can be used as a post and may vary even on a frame.

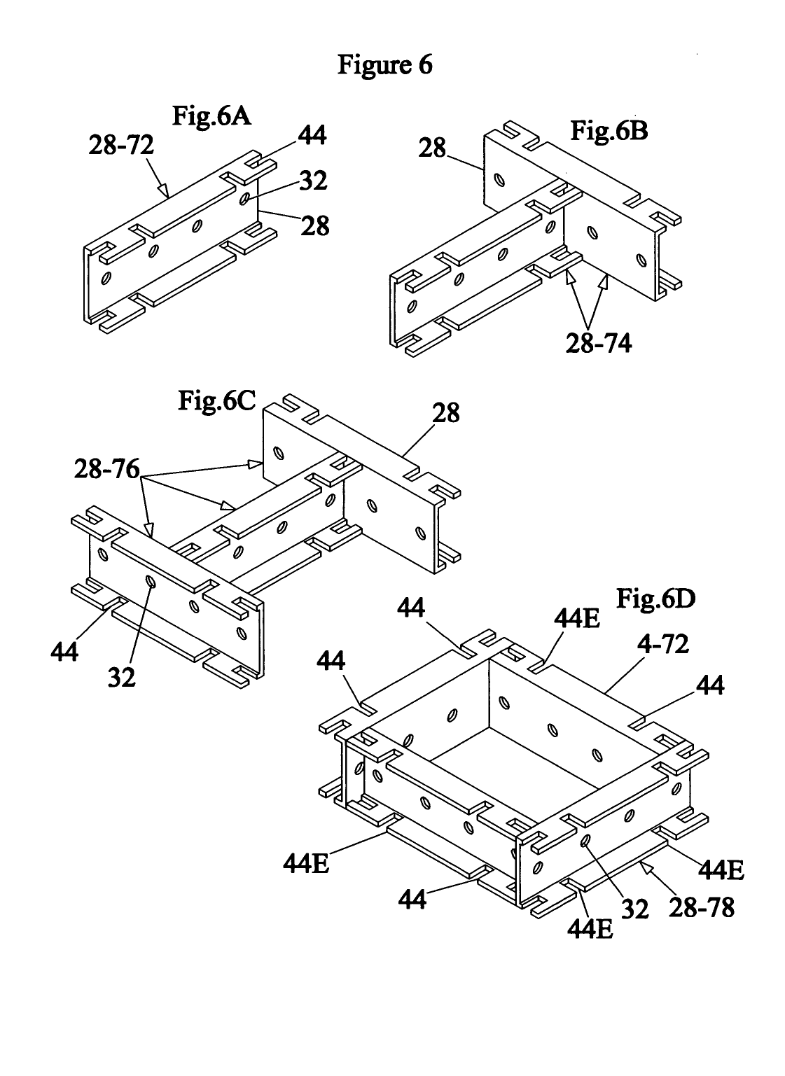

E. Additional cutout positions for extender Compound Post Union Frame 28 FIG. 6D model 78

46.

A. U-bolts and nuts used on all frames 28, they are used to attach T-post 46C to Frame 28 FIGS. 1G-9

B. Bracket for open end of U-bolt 46A FIG. 1H

C. Enclosure fence type T-post 46C is used to represent any post, pipe, rod or tubing 46C FIGS. 1J-9

48. Plate FIGS. 2-3-4-5-7-8-9

Steel square, rectangle, circle or a trapezoid plate used as a frame 28 or as half of a frame 28;54-56-58-60-62. All of these plates 48 have rectangle cutouts 44 in a pattern for attaching items (in examples and drawings, T-post 46C) will be used to form a compound post 80.

50. Steel clamp connector and/or spacer FIG. 1

A.1 Bolts and Nuts

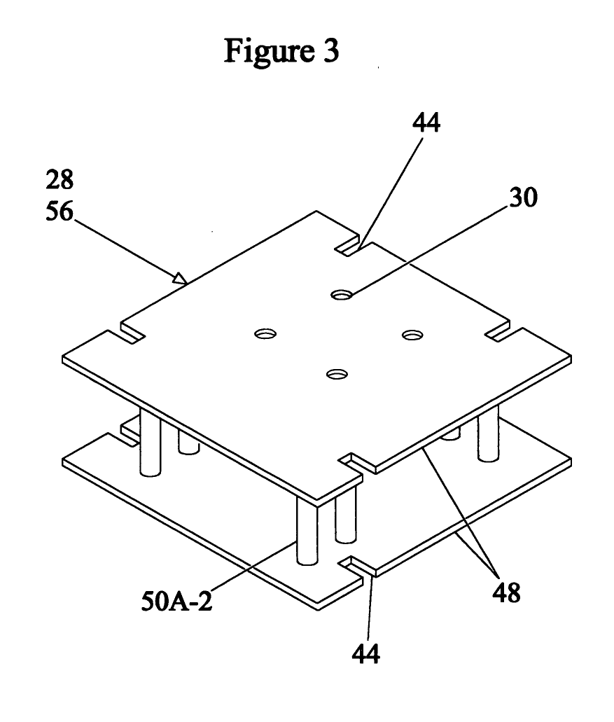

A.2 Two rods FIG. 3

B. Flat Plate FIGS. 2-7-8-9

C. Channel Bar FIGS. 1D-5

D. Angle Plate FIG. 1E

E. Cutouts 34-50E FIGS. 4-1F

Steel clamp connectors 50B-C-D are about one fourth the size of Frame 28, plate 48 for Connecting and separating two plates 48 of a Frame 28 and or for attaching U-bolt 46A to hold small post 46C. The clamp connector 50 can be flat 50B, angle 50D or channel bar 50C, steel rod 50A2 welded to Frame 28, plate 48 bolts and nuts 50A1 are bolted to Frame 28, plate 48, or cutouts 34-50E. Connectors can be 50A1-2 or 34-50E can be bracketed with T-post 46C, by clamp 46A and drawn tight and secure, connector 34-50E is made by cutting three sides of a rectangle with a small square on the top-40 and bending out-42.

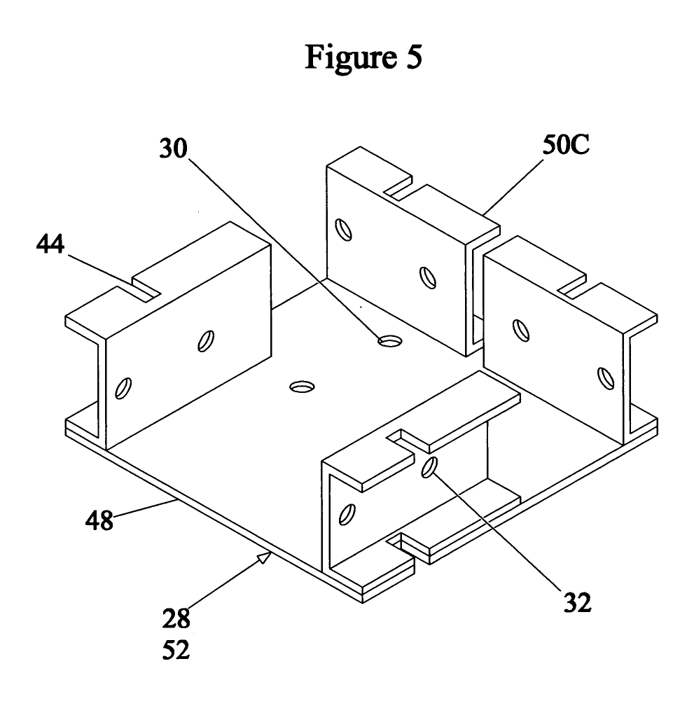

52. Single flat plate frame 28 FIG. 5

Is a single flat plate 48, frame 28 with four cutouts 44 near each comer for attaching T-post 46C. Angle 50D or channel bar 50C connector is welded and centered on each cutout 44 to clamp 46A T-post 46C to the frame 28, plate 48 forming a single plate 48, frame 28 compound post 80.

54. Frame 28 FIG. 2

A frame 28 of two identically cut and drilled plates 48 rectangle or square connected by four connectors 50A1 and 2-B-C-D-E to form a frame 28 for a multiple post 46C compound post 80. FIG. 2 is with connector 50B, FIG. 3 is with 50A2 rod to form Frame 56.

56. Frame FIG. 3

A frame of two (square shown in FIG. 3), rectangle, trapezoid or circle plates 48 with cutouts 44 and brace holes 30. The plates 48 are held in place by pairs of welded rods 50A-2 centered on cutouts 44. A clamp 46A surrounds the bolts 50A1, rods 50A2 or 34-50E and the T-post 46C in cutout 44 to form the compound post 80.

58. Frame 28 FIG. 4

A frame 28 (square shown in FIG. 4) is made with this press and cut and bend 42 method. The rectangle would be the square extended between the end of plate 48 with two cutouts 44 and the end with none. This press cut and bend 42 method can be applied to all frames. The cutouts 44 in the near comer pattern (two on one side near each comer, none on the opposite side, one each on the other two sides near the comers farthest from the side with cutouts 44). There are four for the square 54, three for the trapezoid 60 and six evenly spaced around the round 62. The side with two cutouts 44 has centered on the left cutout 44 and position so that the uncut base is 13 mm-one half inch farther from the edge than the depth of the cutout 44 two rectangular bottle shaped partial cutouts 34-50E. The partial cutouts 34-50E are approximately 25 mm-one inch wide and 75 mm-three inches long. The cut end of the cutout 34-50E is made to have a protrusion in the middle that is 25 mm-one half inch by 25 mm-one half inch. The diagonal comer has the same cutouts 34-50E. The right comer on the side with two cutouts 44 have centered on them and 25 mm-one half inch farther from the edge two, 25 mm-one half inch by 3 mm-one eight inch cutouts 38 that are parallel to the side. The left diagonal comer has the same. The bottle shaped cut and bend out 50E-34 are bent 42 upward on two identical plates. Then connectors 50E-34 are turned to one another and plate 48 is turned to align bottle 50E-34 top 40 with hole 38 for insertion and bending of top 40 to form compound post 80 frame 28. This method of construction with lighter material also forms a puzzle for entertainment.

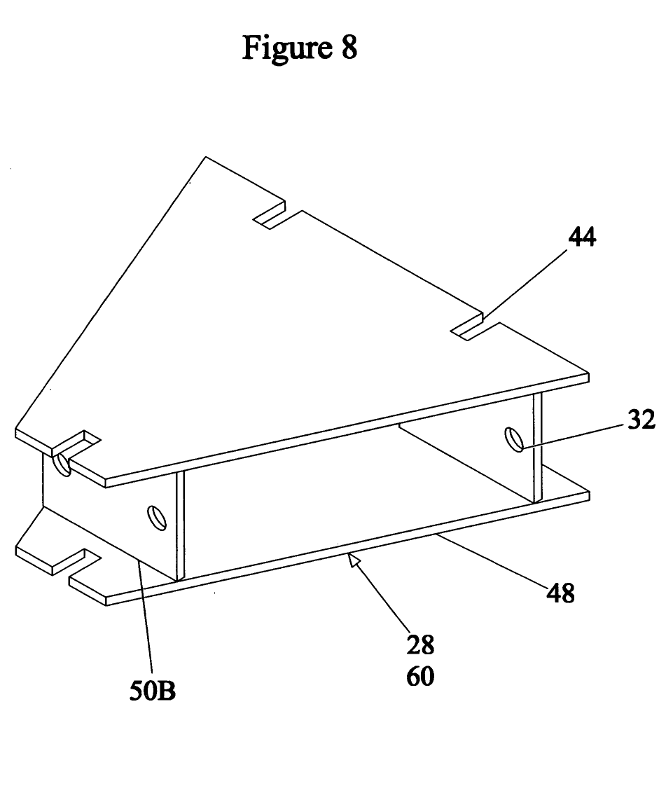

60. Frame 28 Trapezoid FIG. 8

A frame 28 (FIG. 8) of two identically cut and drilled trapezoid plates 28 connected by any connector 50A1 and 2-B-C-D-E to form a triangular three post 46C compound post 80, frame 28. FIG. 8 is shown with flat connector 50B.

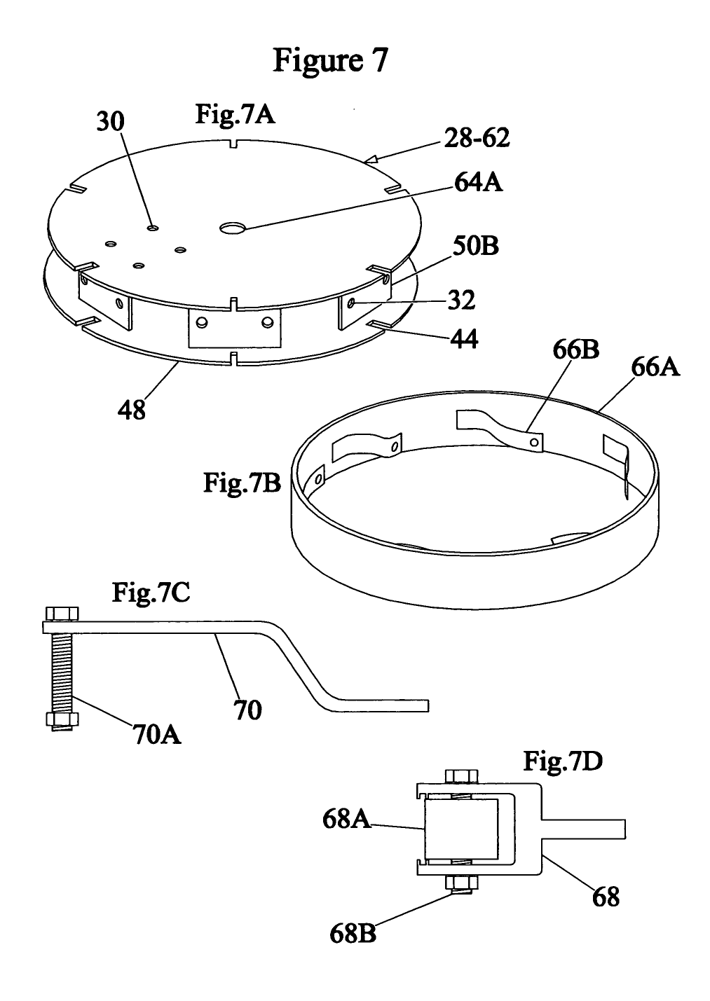

62. Frame 28 Round FIG. 7A

Is a round frame 28 constructed of two identically cut and drilled plates 48 of weld (shown in FIG. 7A) or press cut methods (square shown in FIG. 4) is connected by any of the connectors 50A1 and 2-B-C-D-E. This frame 28 has a larger bolt hole 64A in the center for mounting a 360 degree turning gate, observation stand, antenna and other items.

64. A. FIG. 7A a large hole in the center of frame 62 for mounting items for use.

66. Track FIG. 7B

A. A round flat metal open wheel like track of a size to encircle Compound Post 62-80 with inside centered connection straps 66B for bracketing and locking wheel 66A too frame 62 assembled with post 46C to carry gates attached at bottom hinge in 360 degree turn around compound post 80.

B. Strap of steel flat metal with bolt hole 32 in one end and the other end is welded to track 66A. It is even spaced and centered on the inside of the track 66A. Hole 32 is connected by double nut to clamp 46A on assembled Compound Post Union Frame 62-80.

68. Yoke FIG. 7D

Yoke with center wheel 68A and wheel shaft 68B for holding bottom of gate on to the 360 degree track 66A of compound post 80, frame 62 when used to hold a gate or revolving items.

A. Wheel to carry weight of gate and move around compound post 80 of frame 62. One of 68 is needed for each gate and possible more for other items..

B. Bolt shaft to hold wheel 68A on yoke 68 as the wheel 68A moves on track (66A) around compound post 80. Shaft 68B also pulls together the yoke 68 to catch track 66A in the hooked notched end of yoke 68.

70. Gate arm FIG. 7C

Top gate arm of flat steel or round bar with a bolt hole 64A in one end for the gate to turn freely on and attach with bolt assembly 70A in hole 64A, to compound post 62 frame. The other end attaches to the top hinge end of a gate.

A. large bolt and nut assembly for mounting items on frame 62 Compound Post 80 FIG. 7C

72. Channel bar Frame FIG. 6A

Frame 28 constructed from a single length of channel bar steel or pressed steel in shape of channel bar with cutouts 44 and holes 32 for mounting T-post 46C. When mounting post 46C on ends of 72-74-76 U-bolt clamp 46A will be used and connect into a hole 32 made for it or the holes 32 for near comer connect 44 already there. This frame 28 can be used to hold two to four post for a compound post 80 and can be used as steps of a ladder on other compound post 80.

74. T shaped Frame 28 FIG. 6B

Frame constructed by attaching one channel frame 72 to the middle back (flat side) of another channel frame 72 to form a T shaped frame 28.

76. I shaped Frame 28 FIG. 6C

Frame 28 constructed by attaching one channel frame 72 by its flat middle back to a frame 28-74 to form a capital I shaped frame 28.

78. Box channel Frame 28 FIG. 6D a frame 28 constructed by joining four frame 72 end to end with concave side out to form a box channel frame 28. All channel frames 72 may not have bolt holes 30 for brace post 46C but instead be embraced by connector clamp 46A with cross clamp brackets 46B. All channel frames do have bolt holes 32 centered on cutouts 44.

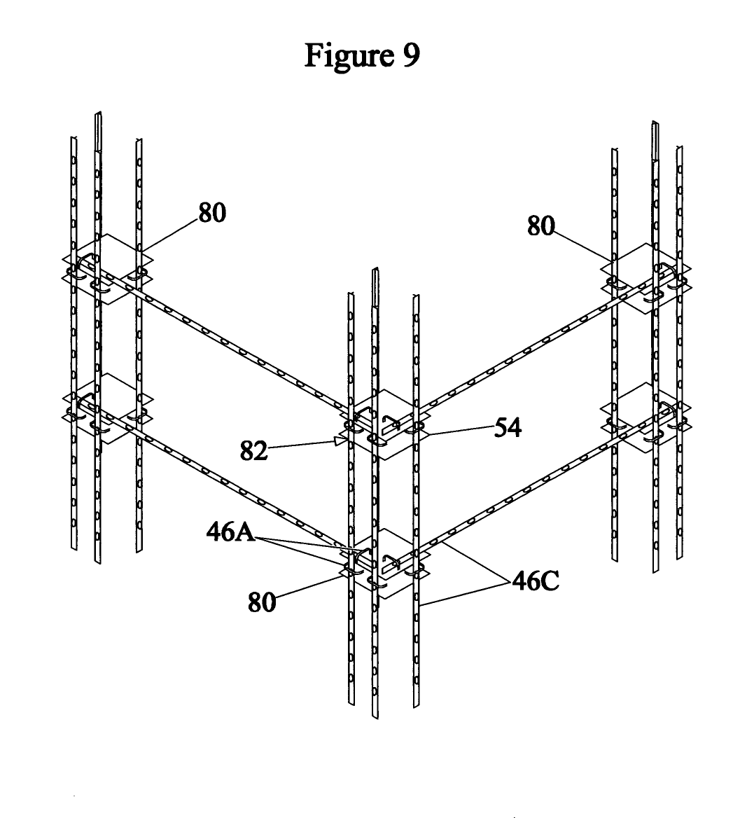

80. Is an assembled Compound Post 80. FIG. 9 is of three Compound Post 80

82. FIG. 9 is three assembled compound post 80 connected by four T-post 46C as braces.

The object of this invention (Compound Post Union Frame 28) is to have a means to securely fasten items (post, pipe, rods or tubing) together to add or multiply their strength in a single Post 80.

The Compound Post Union Frame 28 FIGS. 2 through 9 can be made in several shapes and sizes for a variety of uses, price and strength requirements. The Post Union Frame 28 can be made with different production methods. The single channel bar 72, two channel bars 74-72 joined to form a capital T, three channel bars 76-72 joined to form a capital I, and four channel bars 78-72 joined concave side out and end to end to form a square or rectangle 78-72, may be the most versatile and least expensive. Square Post Union Frame 54-28 of flat steel plate 48 trapezoid 60-28 of flat steel plate 48 and round 62-28 of flat steel plate 48, have been made and tested. The rectangle not shown would be the same as the square 52,54,56 and 58 with added length between the sides with two cutouts 44 near the comers for small post and the side with none. The other two sides each have one cutout 44 near the comer next too the side with none. This square pattern and the round pattern of evenly spaced cutouts 44 when increased in number forms the extension Post Union Frame 28-44E FIG. 6D. All Post Frames 28 have cutouts 44 and holes 30 in a pattern for attaching items such as T-Post 46C. The pattern depends on the items to be attached and the clamp 32 used.

The 72 through 78 frames 28 may not have holes 30 for U bolt clamps 46A to attach cross braces 46C, gates or other items all of the others may need them. The U-bolt, bracket and nuts 46A embrace the T-post 46C, gate connector or other items to the channel bar 72 itself. The single flat plate 48 frame 52 is not as strong as the double plate 54,56,58,60, 62 or the channel bar 72 models.

The first descriptions used and the construction methods described for Compound Post Union Frame 28 will be a general description for double plate 48 cut and weld frames made for four T post—46C of the enclosure fence. Rigid strong material is required to make the Compound Post Union Frame 28 and recycled steel will be used in the description.

MAKING THE SQUARE CUT AND WELD COMPOUND POST UNION FRAME 54-28 FIG. 2 This is a description for making one version of the square variation of the Compound Post Frame 28 use approximately 3 mm-one eight inch steel plates approximately 21 cm-eight inches square, have four cutouts 44 approximately 20 mm-three fourth inches deep, approximately 7 mm-one fourth inch wide to accept the unnodule side of T-post 46C two of these cutouts 44 are on one side approximately 38 mm-one and one half inches from the comers. The opposite side does not have cutouts 44. The adjoining sides each have a cutout 44 approximately 38 mm-one and one half inch from the far comer of the side with two, four holes 30 are drilled near the center of the plate 48 the holes 30 are in a pattern to match the U-bolt 46A to be used. The size and spacing of the holes 30 will accept the U-bolt 46A and be arranged for use of two U-bolts 46A in any direction.

Four approximately 3 mm-one eight inch steel plates 50B,C,D approximately 63 mm-two and one half inches one way and the other the width of the U-bolt 46A to be used plus 25 mm-one inch. The U-bolt 46A width side has holes 32 drilled for the U-bolt 46A centered on the 63 mm-two and one half one way and centered for the U-bolt 46A to align with T-post 46C cutouts 44 on the larger plate 48 when small plate 50 B,C,D is welded at a ninety degree angle connecting both plates 48.

ASSEMBLE: Place two frames 28, two cutouts 44 sides up on flat surface spaced about 60 cm-two feet apart. Lay two T-post 46C unnodules side in cutouts 44 position, to leave length of T-post 46C above top of frame 28 to operate single post, T-post 46C hammer. Insert U-bolts 46A around T-post 46C and into frame connectors 50 B,C,D attach nut fasteners 46A to firm but light torque, rotate assembly and repeat post 46C assembly for remaining two post 46

INSTALLING: Compound Post 80 in fence, place Compound post assembly 80 square with fence line and two post 46C side nearest line of fence. Relieve torque tension on two clamps 46A of one post 46C and drive into ground with T-post 46C hammer. Torque clamps 46A to secure if frames 28 are at desired level or light if frame 28 will be adjusted when all four post 46C have had this procedure accomplished. The farm implement for pounding large post in the ground, that is on the market should also work, Disclosure 541480 Nov. 6, 2003 UNITED T-POST HAMMER may have to be used when installing Compound Post 80 of a height above average reach.

MAKING THE PRESS CUT FORM OF COMPOUND POST UNION FRAME 58-28 FIG. 4 Press cut two 3 mm-one eight inch steel plates 48 approximately 21 cm-eight inches square, have four cutouts 44 approximately 20 mm-three fourth inch deep approximately 5 mm-three sixteen inch wide to accept the unnodules side of T-post 46C. Two of these cut outs 44 are on one side approximately 38 mm-one and one half inch from the comers. The opposite side does not have cutouts 44. The adjoining sides each have a cutout 44 approximately 38 mm-one and one half inch from the far comer of the side with two. The side with two cutouts 44 has centered on the left cutout 44 and position so that the uncut base is 13 mm-one half inch farther from the edge than the depth of the cutout 44 are two rectangular bottle shaped partial cutouts 34-50E. The partial cutouts 34-50E are approximately 25 mm-one inch wide and 75 mm-three inches long. The cut end of the cutout 34-50E is made to have a protrusion in the middle that is 13 mm-one half inch by 13 mm-one half inch. The diagonal comer has the same cutouts 34-50E. The right comer on the side with two cutouts 44 have centered on them and 13 mm-one half inch farther from the edge, two 13 mm-one half inch by 3 mm-one eight inch cutouts 38 that are parallel to the side. The left diagonal comer has the same. The Post Frame 28 to be made for brace post 46C will have four holes 30 drilled in right comer farther from the edge than the cutouts 38. The cutouts 38 and 34 will serve as connectors 50E for the two plates 48 and have in the center of the rectangle part a hole 32 to receive one side of the U-bolt clamp 46A with nuts only.

Holding the two plates 48-58 in a side by side position so that they are identical with bottle shaped connector 50E bent up, turn as closing a book. The top 40 of bottle shaped connectors 34-50E go into cutouts 38 and are bent to lock the two plates 48-58 to together. This press and cut design can be used on all Post Frames. The rectangle Post Frame 28 not shown is identical though extended to all square models and same construction apply.

MAKING THE COMPOUND POST UNION FRAME 56-28 FIG. 3. The Post Frame 56 is made with double plates 48 of any shape. The cutout 44 pattern is the same as those used on other Post Frames 28 of the same shape. The connect rod 50A is welded to the plate 48 to establish desired distance between plates 48 centered on the cutout 44. The bolt 50A or connector 50E can be used in the same method. Assembly requires a U-bolt, nuts 46A, and bracket 46B to embrace connector rod 50A or E and T-post 46C in cutouts 44.

MAKING THE ROUND COMPOUND POST UNION FRAME 62-28 FIG. 7 The Post Frame 62 is made of two round plates 48 size and number of post 46 can vary for use. Description will be for a 30 cm-one foot diameter circle plate 28 with six evenly spaced cutouts 44 around outer edge. The Post Frame 62 may have drilled U-bolt holes 30 if braces are to be attached. The Post Frame 62 will have a bolt hole 64A drilled in the center in both plates 28 to accept a bolt 64B. Post Frame 62 can be made using 50A1 and 2 B,C or E as connectors centered on cutouts 44. Post Frame 62-80 when used as a hunting or observation stand would have frames 62 at spaced points chosen and one at the top of the tower for a revolving seat. Near the top of the tower 80 or attached to the revolving seat would be a standing platform with camouflage cover. Post Frame 62-80 when used as a 360 degree gate post can mount several gates to be used in a livestock circular pen for livestock to be loaded, medicated or separated. The Post Frame 62-80 can be used for entrances where the gate needs to open in either direction and swing back until something other than the post 80 itself stops the gate.

ASSEMBLY: Post Frame 62 is similar to all other Post Frames 28. The frame forms a channel with a U-bolt 46A exerting pressure in the middle area of the channel to lock items to Post Frame 28. Post Frames 62 for 360 degree revolving gate requires the addition of a track 66A to encircle Post Frame 62 on a lower level frame 62 and is attached by straps 66B welded on the track 66A, to the U-bolts 46A holding T-post 46C as part of Post Frame 62-80. The gate on the bottom will have one end of a yoke 68 shaped attachment in the place of the normal hinge assembly. The yoke 68 may have to be a double yoke 68 for gates used in heavy stress. The yoke 68 may have a channel configuration at the ends of the forks of yoke 68 to prevent the gate being lifted on the non hinge end and a revolving roller, wheel 68A device to carry the weight of the gate. The wheel would be mounted on a bolt or shaft 68B that also adjust forks of yoke 68 to track 66A. The gate will have one end of a arm 70 attached to the hinge end ,top of the gate. The other end with a hole in it will curve to align with a large bolt 64B in the hole 64A in the center of Post Frame 62-80 at the top of post 80. The bolt 64B end of 70 may need to vary in shape on each when used with additional gates to allow for short overlapping moves during operation. The yoke 68 on low attachments does not have this circumstance.

MAKING THE TRAPEZOID 60 COMPOUND POST UNION FRAME FIG. 8 This is a description of the three post 46C trapezoid 60 Post Frame. The narrow side of the trapezoid has a connector cutout 44 and is wide enough to accept a connector 50. The opposite side has the two cutout 44 connectors approximately 38 mm-one and one half inch from the comer. The other two sides have none. When using connectors 50A,B,C or D, two identical plates are attached. When using connector 50E one plate 48 has cutouts 34 and the other has cutouts 38. Placement of connectors are centered on cutout 44.

MAKING THE FLAT PLATE 28 ANY SHAPE 52 COMPOUND POST UNION FRAME 28 (square in FIG. 5): The Post Frame 52 is made with a single plate 48 of any shape. The Post Frame 52 can use only connectors 50C or D. The connectors are welded so that the angle of 50C or D can have cutout 44 and be positioned the same as in Post Frames 54,56,58,60 or 62.

MAKING THE CHANNEL BAR COMPOUND POST UNION FRAME 72-28 AND THE JOINED FORM COMPOUND POST UNION FRAMES 74,76 AND 78 FIG. 6 This is a description of four of the unlimited number of models that can be created from the channel bar 72. The not described models will be created when additional channel bar 72 are connected.

A basic two rod or post 46C model would only require 15 cm-six inches of bar or less when using only ends and would have cutout 44 38 mm-one and one half inch from each end, and centered on each end. The holes 32 for T-post 46C clamps 46A attachment will be in the third side centered on the cutouts 44. This pattern has unlimited possible designs for post 46C and channel bar additions in other direction. Channel bar Post Frame 72-28 can be joined to a Post Frame 72 by positioning end to end, along the flat side or the narrow channel end. The connection can be made by welding or by cutouts 38 and protrusion 40 of the channel bar.

The Post Frame 72-28 FIG. 6A is a length of channel bar with cutouts on both channel sides in matching pairs. An optional pair of cutouts 44 can be made in the ends. The end post 46C connection can be connected by reversing the U-bolt 46A connection from other Post Frame 28. A single hole 32 in the middle of channel bottom with a U-bolt 46A inserted through and out to embrace a T-post 46C and using a bracket 46B and nuts to complete and secure the embrace.

Post Frame 74 FIG. 6B is made by connecting the end of one Post Frame 72 to the middle of the flat side of another to form a T.

Post Frame 76 FIG. 6C is made by connecting one Post Frame 72 by its flat side in the middle to a Post Frame 74 to form a I.

Compound Post Union Frame 78 FIG. 6D is made by connecting four Post Frames 72 with channel side out end to end to form a square or rectangle. FIG. 6D has Post Frame 78 with extender cutouts 44E. The Post Frame 78 requires less additional change to become a Post Frame extender model due to brace connection without holes 30 and extra cutout 44 on Fames 72.

In conclusion, having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for and disclosure pertinent to the practice of this invention. Numerous variations are still possible, while adhering to the inventive concept. Such variations are contemplated as being a part of the present.

1. A frame for joining T-Post, rods, pipe, post tubing or any elongated material together to form a post or tower. The device is formed from rigid material of pipe or channel shaped material forming frames. Flat rigid material of round, trapezoid, square or rectangle material forms a frame. Size, shape, number of items joined and material of all components may vary to form variants of post and frame.

2. The said device of claim one has cutouts in or on the material for attaching rods, post, pipe, tubing or any elongated material.

3. The said device of claim one has bolt holes of variable size to attach items both in making said device and in operation.

4. The said device of claim one includes clamping devices.

5. The devices of claim one connecting elongated items form a brace unit for fences and when used at several levels, forms a fence of itself.

6. One form of the holes of claim three with clamping device add items in line of elongated material such as antennas or gate support and at ninety degree to said line braces and connectors.

7. The round device of claim one attaches a three-hundred sixty degree, turning attachment with the use of holes of claim three.

8. The said device of claim one in some of its variants of material, shape and size requires connectors of rigid material with or without holes of claim three.

9. The device of claim one in some of its forms will join two of its rigid material forms of variant shape and size by bend out parts of one too the other.

10. Additional cutouts of claim two extend the elongated material.

11. The device of claim 9 of the bend out model forms a sample pattern for use in making entertainment puzzles of lighter material.

12. A fencing system forming a braced fence with the use of claim 1.

13. The device of claim 1 can be used to form and as steps of a ladder for climbing fences, towers or stands.

14. The said device of claim 1 with extender of claim 10 forms system for taller post, tower or horizontal barricades.

15. The joining of items to form a post, tower or barricade.

16. The joining of two or more items with a channel clamping system.

17. A system using claim 1 for framing of steel in cement construction.
